(19) United States
(12) Patent Application Publication
(10) Pub. No.: US 2002/0151537 A1
(43) Pub. Date: Oct. 17, 2002

(54) Composition and method for treating erectile dysfunction and reducing fibrosis in erectile tissue of the human penis

(76) Inventor: W. Jerry Easterling, San Antonio, TX (US)

Correspondence Address: DAVID G. HENRY, 700 Texas Center, P.O. Box 1470, Waco, TX, 76703-1470, US

(21) Appl. No.: 10/044,288

(22) Filed: Oct. 24, 2001

Related U.S. Application Data
(63) Continuation-in-part of application No. 09/411,175, filed on Oct. 1, 1999, now Pat. No. 6,353,028.
Application No. 09/411,175 is a continuation-in-part of application No. 09/128,103, filed on Aug. 3, 1998, now Pat. No. 6,031,005.

(51) Int. Cl. A61K 31/554; A61K 31/44; A61K 31/137

(52) U.S. Cl. 514/211.07; 514/355; 514/648

(57) ABSTRACT

The invention is of a topical medicament and associated methodology for use thereof, through the use of which fibrosis of the elastic tissues and muscles of the human penis is reduced, thereby improving erectile function and/or reversing erectile dysfunction. One or more calcium channel blocker agents serve as the primary active ingredient of the present compositions, with carrier agents facilitating non-invasive transdermal delivery of the calcium channel blocker(s) to fibrotic tissues of the penis.

### CITATION TO PRIOR APPLICATION

This is a continuation-in-part with respect to U.S. application Ser. No. 09/411,175, filed Oct. 1, 1999, which, in turn, was a continuation-in-part of U.S. application Ser. No. 09/128,103 (now U.S. Pat. No. 6,031,005), from which application and its parent application priority is here claimed under 35 U.S.C. §120.

### BACKGROUND OF THE INVENTION

1. Field of The Invention

Applicant's invention relates to medicaments and treatment procedures relating to erectile dysfunction and poor erectile quality caused by fibrosis of erectile tissue which, in turn, results in loss of length and girth of the human penis.

2. Background Information

Fibrosis is a common response to numerous conditions, including but not limited to the following:

Aging

Tissue necrosis

Trauma or Injury

Connective Tissue Disease

Hypertension

Diabetes

Arterial Insufficiency

Atherosclerosis

Fibrosis of cavernosal smooth muscle tissue results in the loss of elasticity of this smooth muscle tissue, thereby interfering with the normal expansion of the cavernosal chambers when filled with arterial blood during erection. Therefore, a partial penile erection or no erection may occur.

Erectile dysfunction due to fibrosis is common from the fifth through the eighth decade of life, while the capacity for erection often is not changed. A hypothesis of the present inventor was that, because fibrosis underlies certain forms of erectile dysfunction, his topical, calcium channel blocker medicaments might be efficacious in treating such forms of erectile dysfunction as arise from fibrosis because of the common causative roots of fibrosis-related erectile dysfunction and Peyronie's disease—excessive formation of connective tissue.

As discussed below, the inventor's hypothesis proved correct.

### SUMMARY OF THE INVENTION

It is an object of the present invention to provide a novel medicament useful in the treatment of fibrosis of muscle tissue such as contributes to erectile dysfunction.

It is another object of the present invention to provide a novel medicament useful in the treatment of fibrosis of erectile tissues such as contributes to degradation in quality of erection in men, even though not to the extent necessarily recognized as erectile dysfunction.

In satisfaction of these and related objectives, Applicant's present invention provides a topical medicament and associated methodology for use thereof, through the use of which fibrosis-based erectile dysfunction or diminishment in erectile quality may be effectively, cost effectively, and painlessly treated. The topical medicament is formulated to non-invasively, and transdermally deliver calcium channel blocker agents to elastic tissues of the human penis. When used for a period of weeks or months, users report significant remediation of erectile dysfunction, or, if not so identified as erectile dysfunction in the first place, increase in length and/or girth of the penis upon erection.

The invention, although exemplified by specific embodiments which are based upon, or rely on the use of specific calcium channel blockers, is not limited to such species. Rather, observations by the present inventor indicate that when coupled with a suitable carrier for transdermal delivery, all thus-far-evaluated calcium channel blockers effect reduction of fibrotic tissue disorder symptoms. Therefore, the true scope of the invention encompasses preparations and methods of use facilitating or involving the use of transdermal application of calcium channel blockers in the treatment of fibrotic-related erectile dysfunction or diminishment.

All observations of efficacy of the present compositions and methods arise from physician-supervised and prescribed treatment regimens involving use of the medicaments of the present invention. In most cases to date, use of the present medicaments and prescribed treatment regimens were in connection with the treatment of the more serious condition of Peyronies Disease.

Recent studies by the present inventor and colleagues (as discussed below) involve the treatment of erectile dysfunction though use of the medicaments taught herein. These studies indicate that use of topical calcium channel blocker medicaments taught herein are highly effective in treating erectile dysfunction which appear to relate to fibrosis of cavernosal smooth muscle tissue.

In a experimental study, 142 patients reporting decreased quality erections were treated with topical Verapamil (80 mg/mL). One hundred thirty four (94.3%) experienced improvement of erectile rigidity and/or improvement of penile girth upon erection. These patients applied 0.5 mL of the Verapamil topical compound to the entire shaft of the penis twice a day. The length of treatment varied from one to several months, with the mean treatment period being 3.5 months. Patients were either examined or interviewed and counseled at least every thirty days in order to evaluate progress and monitor side effects. The only side effect reported was contact dermatitis in less than one percent of the patient population. This side effect was easily controlled with topical corticosteroids.

Similar results have been observed in patients treated with a topical Nifedipine compound (40-60 mg/ml) or a combination of Verapamil and Nifedipine.

Formulations for the topical Verapamil, Nifedipine, and combination Verapamil-Nifedipine are identical to those provided herein with respect to Peyronie's Disease and the other discussed connective tissue disorders.

Upon initial suggestion of the present compositions and methods for use in treating fibrotic or connective tissue disorders, the present inventor experienced, at the hands of experienced practitioners in the field, expressions of serious doubt as to efficacy, and, in some cases, outright ridicule. The stated basis for such initial doubts and criticisms related to the fact that intralessional injections of precisely the same substances (calcium channel blockers) in the attempted treatment of Peyronie's disease and similar fibrotic tissue disorders had yielded very sporadic and limited results. The present inventor was told repeatedly by those experienced in the treatment of fibrotic tissue disorders (Peyronie's disease, in particular) that a topical preparation based on calcium channel blockers could “never” have efficacy in view of the failure of injections of the same substances directly into the plaques of these conditions. As mentioned previously, actual experience teaches that the strikingly counter-intuitive effect of the compositions and methods of the present invention in the treatment of fibrotic tissue disorders is very much real—a fact born out by the substantial commercial success of the present medicaments in a very short time (less than a year at the time of preparation of this application) and with no commercial marketing whatsoever, as well as the immediate replacement of existing treatment regimens by the medicaments and methods of the present invention by numerous medical practitioners.

### DETAILED DESCRIPTION OF THE PREFERRED EMBODIMENT

In the preferred embodiment of the present medicament, and in the medicament upon which the associated method are based, the primary active ingredient is Verapamil Hydrochloride, USP (a diphenylalkylamine). However, it should be understood that other calcium channel blockers (topically applied in a similar composition) provide similar results. With certain patients, combinations of channel blocker agents seem to have an even greater efficacy than the single, Verapamil agent. Other such calcium channel blockers include benzothiazepines (Diltiazem, for example), dihydropyridines (Amlodipine, Felodipine, Isradipine, Nicardipine, Nifedipine, Nimodipine, or Nisoldipine), and the fast sodium inward channel inhibitor—Bepridil. Diltiazem in particular, has proven effective when substituted for Verapamil, particularly for patients with a demonstrated skin sensitivity to Verapamil. Appropriate dosage substitutions when substituting one particular calcium antagonist for another (Verapamil for Diltiazem, for example) will be made in same manner as if such agents were being interchanged for their existing, more conventional uses). Likewise, combining multiple calcium antagonists will result in similar dosage considerations, as will be apparent to persons skilled in the art.

I. Formulation

In evaluating deterioration problems with the prior embodiments of the present inventor's medicaments, the present inventor may get the following observations and/or came to certain conclusions:

1. Air is being entrained into the materials at all stages of formulation.

The ethoxydiglycol reagent is reacting with the air and forming byproducts including but not limited to aldehydes, peroxides, and free radicals which cause drug crystallization and subsequent loss of therapeutic potency. Additionally, these byproducts can cause skin irritation.

Verapamil is a chemical derivative of papaverine. Papaverine, in the presence of heavy metals, will deteriorate rapidly. The verapamil formulations may be affected by the presence of heavy metal ions that originate from the mixing containers or equipment.

Based upon these conclusions, the present inventor made the following basic changes to his prior formulations and preparation steps:

1. Butylated hydroxytoluene (BHT), NF. BHT is added, and serves as an antioxidant to counteract any reaction with entrained air.

2. Nitrogen, NF, is used to purge all containers during chemical addition and mixing. Every ointment tube is purged just prior to filling and sealing. The nitrogen serves as a replacement for entrained air and is non-reactive with the components.

3. A “non-reactive” glaminate ointment tube is used so that no reaction occurs with the ointment tube.

4. Edetate disodium, USP is added to the gel formulation and serves as a chelating agent to bind any heavy metal ions and prevent reaction of same.

b 5. Propylene glycol, USP has been added as an additional drug solvent and skin absorption enhancer.

The result of making the preceding changes to the prior gel formulations is a gel which is stable over periods of many months, even after undergoing formal, rigorous stability studies by an independent pharmaceutical laboratory. Patient evaluations indicate that the change in formulation has in no way negatively affected efficacy and, and fact, appears to have somewhat enhanced such efficacy.

II. Preparation

The now-preferred Verapamil-based gels of the present invention (in exemplary 10% and 15% percent strengths) may be prepared according to the following disclosure and protocol, with variations appropriate to a desired scale of production as will be apparent to persons skilled in the production of pharmaceutical preparations:

A. Constituents of Preferred Embodiment
of Topical Verapamil Gel 10% and 15%

| Ingredients | 10% (% W/W) | 15% (% W/W) |
|---|---|---|
| Verapamil | 10.0 | 15.0 |
| Ethoxydiglycol | 14.0 | 19.5 |
| Propylene Glycol | 0.5 | 0.5 |
| Butylated Hydroxy Toluene (BHT) | 0.1 | 0.1 |
| Lecithin Soya Granular | 13.1 | 13.1 |
| Isopropyl Myristate | 13.1 | 13.1 |
| Sorbic Acid | 0.09 | 0.09 |
| Pluronic F127 | 9.8 | 11.6 |
| Potassium Sorbate | 0.15 | 0.12 |
| Disodium Edetate | 0.01 | 0.01 |
| Purified Water | 39.15 | 26.88 |

B. Topical Verapamil 15% (To Make 3000 Gm).

| Ingredients | Quantity | |
|---|---|---|
| Verapamil HCI USP | 450.00 | Gm |
| Ethoxydiglycol Reagent | 585.0 | Gm |
| Lecithin/Isopropyl Myristate Solution | 790.0 | Gm |
| Butylated Hydroxytolune NF (BHT) | 3.0 | Gm |
| Edetate Disodium USP | 0.30 | Gm |
| Propylene Glycol USP | 15.0 | Gm |
| Pluronic Gel 30% | 1,156.7 | Gm |

Instructions: Dissolve verapamil in ethoxydiglycol and propylene glycol with the aid of heat (90-100 degrees C.). Stir during this dissolving step. When the solution is clear, weigh to ascertain the amount of evaporation. Add the amount lost to evaporation back as ethoxydiglycol. Immediately add the lecithin/isopropyl myristate and BHT and stir well. Weigh the PLO 30% into a plastic container, add edetate disodium and stir gently to dissolve edetate disodium. Avoid foaming with stirring. Gently add the verapamil phase to the PLO phase, avoiding the incorporation of air. Stir for 10 minutes using a 3 inch mixing blade at 3100 rpm. Dispense in 30 Gm glaminate ointment tubes.

C. Topical Verapamil 10% (To Make 3000 Gm).

| Ingredients | Quantity | |
|---|---|---|
| Verapamil HCI USP | 300.00 | Gm |
| Ethoxydiglycol Reagent | 420.0 | Gm |
| Lecithin/Isopropyl Myristate Solution | 790.0 | Gm |
| Butylated Hydroxytolune NF (BHT) | 3.0 | Gm |
| Edetate Disodium USP | 0.30 | Gm |
| Propylene Glycol USP | 15.0 | Gm |
| Pluronic Gel 30% | 1,471.7 | Gm |

Instructions: Dissolve verapamil in ethoxydiglycol and propylene glycol with the aid of heat (90-100 degrees C.). Stir during this dissolving step. When the solution is clear, weigh to ascertain the amount of evaporation. Add the amount lost to evaporation back as ethoxydiglycol. Immediately add the lecithin/isopropyl myristate and BHT and stir well. Weigh the PLO 30% into a plastic container, add edetate disodium and stir gently to dissolve edetate disodium. Avoid foaming with stirring. Gently add the verapamil phase to the PLO phase, avoiding the incorporation of air. Stir for 5 minutes using a 3 inch mixing blade at 3100 rpm. Dispense in 30 Gm glaminate ointment tubes.

D. Pluronic Gel 20% (To Make 3000 Gm)

| Ingredients | Quantity |
|---|---|
| Pluronic F127 NF (Poloxamer 407) | 600.00 Gm |
| Potassium Sorbate NF | 9.00 Gm |
| Water (Sterile for Irrigation) qs to | 3,000.00 Gm |

Directions: Prepare a pluronic gel by combining the potassium sorbate and pluronic F127 and bringing to a total weight of 3,000 Gm. with cold (refrigerated) sterile water. Make sure that all the granules are wet, and place in a refrigerator. Mixture will form a clear solution over 24-48 hours.

Alternate Procedure: The above mixture can be uniformly mixed with a mixing blade. It will take on the appearance of beaten egg whites. When placed in the refrigerator it will form a clear solution much faster, usually overnight.

The above solution will solidify into a clear gel at room temperature.

E. Pluronic Gel 30% (To Make 2000 Gm).

| Ingredients | Quantity |
|---|---|
| Pluronic F127 NF (Poloxamer 407) | 600.00 Gm |
| Potassium Sorbate NF | 6.00 Gm |
| Water (Sterile for Irrigation) qs to | 2,000.00 Gm |

Instructions: Prepare a pluronic gel by combining the potassium sorbate and pluronic F127 and bringing to a total weight of 2,000 Gm. with cold (refrigerated) sterile water. Make sure that all the granules are wet, and place in a refrigerator. Mixture will form a clear solution over 24-48 hours. Alternate Procedure: The above mixture can be uniformly mixed with a mixing blade. It will take on the appearance of beaten egg whites. When placed in the refrigerator it will form a clear solution much faster, usually overnight. The above solution will solidify into a clear gel at room temperature.

F. Lecithin/Isopropyl Myristate Solution (To Make 3000 Gm).

| Ingredients | Quantity |
|---|---|
| Lecithin Soya Granular | 1,494.0 Gm |
| Isopropyl Myristate NF | 1,494.0 Gm |
| Sorbic Acid NF Powder | 9.90 Gm |

Instructions: Disperse lecithin and sorbic acid in isopropyl myristate. Allow to stand at room temperature until a liquid of syrup consistency forms. Stir well and store in a light protected container.

III. Use of Preparations

The choice of strengths of the topical calcium antagonist gels taught above will depend on the experience of the clinician. Ordinarily, a patent will be started with the lower dosage preparation, and only if the patient fails to respond, or responds more slowing than reasonably would be expected, would the patient be changed to the higher dosage form.

In any event, use of all topical calcium channel blocker preparations of the present inventor's work involves simply applying a thin coating of the gels to the entire penis twice daily. Noticeable results are typically experienced within not more than six months of treatment, if the treatment regimen is faithfully followed by the patient.

Patients should not engage in intercourse with the medication applied as it may irritate the vaginal mucosa.

The patient's progress should be evaluated every 4 weeks to assess changes in plaque, etc. Although some patients respond to the medication during the first month of therapy, others have responded after 2-3 months of therapy. It is important to not miss doses of medication.

Application to the entire penile shaft is important. In initial experimental use of the present medicament, localized application of the gel (solely to areas atop the suspected plaque) effected merely a change in the direction of the previous curvature. Subsequent application to the entire penile shaft in the same patients resulted in complete reversal of symptoms. This phenomena may be explained if plaque, to varying degrees, is present throughout the entire penile shaft, and not just localized to the point(s) of curvature.

During the treatment regimen, each patient's progress should be evaluated, at least every two weeks. If no results have occurred by the end of the 3rd week, the dose should be increased and/or the medicament applied more often than twice daily.

Since calcium channel blockers may be antihypertensive, the patient's blood pressure should be monitored at the physician's office after the first dose of a calcium channel blocker medicament is applied. To date, however, no changes in blood pressure have been noted.

Although the invention has been described with reference to specific embodiments, particularly with respect to the particular active ingredient of the present medicament, this description is not meant to be construed in a limited sense, in particular to limit the scope of the appended claims to cover only those medicaments and associated modalities of treatment which include Verapamil as the calcium channel blocker, the function of which in the area of plaque appears to lie at the heart of the efficacy of the present medicament. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A medicament for use in reducing fibrosis in elastic tissues of the human male comprising:
a transdermal carrier compound for facilitating non-invasive, transdermal delivery of calcium channel blocker agents to internal tissues of the human penis;
a calcium channel blocker agent dispersed in said carrier means.

2. The medicament of claim 1 wherein said calcium channel blocker agent includes one or more calcium channel blocker substances chosen from the classes of calcium channel blockers which are diphenylalkylamines, benzothiazepines, or dihydropyridines.

3. The medicament of claim 1 wherein said calcium channel blocker agent is verapamil.

4. The medicament of claim 1 wherein said calcium channel blocker agent is a benzothiazepine.

5. The medicament of claim 1 wherein said calcium channel blocker agent is a dihydropyridines.

6. The medicament of claim 1 wherein said calcium channel blocker agent is Nifedipine.

7. The medicament of claim 1 wherein said medicament comprises:
verapamil;
a lecithin/isopropyl myristate solution;
butylated hydroxy toluene;
pluronic F127; and
water.

8. The medicament of claim 1 wherein said medicament comprises:
one or more calcium antagonist selected from the calcium channel blocker categories of diphenylalkylamines, benzothiazepines, and dihydropyridines;
a lecithin/isopropyl myristate solution;
butylated hydroxy toluene;
pluronic F127; and
water.

9. The medicament of claim 1 wherein said medicament comprises:
a diphenylalkylamine calcium antagonist agent;
a lecithin/isopropyl myristate solution;
butylated hydroxy toluene;
pluronic F127; and
water.

10. The medicament of claim 7 further comprising:
Edetate disodium.

11. The medicament of claim 8 further comprising:
Edetate disodium.

12. The medicament of claim 9 further comprising:
Edetate disodium.

13. The medicament of claim 10 further comprising:
Propylene glycol.

14. The medicament of claim 11 further comprising:
Propylene glycol.

15. The medicament of claim 12 further comprising:
Propylene glycol.

16. A method for remediating fibrosis of elastic tissues and muscles of the human penis comprising the steps of:
selecting a medicament comprising:
a carrier host agent for facilitating non-invasive, transdermal delivery of a calcium antagonist into internal tissues of the human penis;
a therapeutic dosage of a calcium channel blocker agent suspended in said carrier host agent;
periodically, topically applying a therapeutic dosage of said medicament to the surface of said penis.

17. The method of claim 16 wherein said calcium channel blocker agent is verapamil.

18. The medicament of claim 16 wherein said calcium channel blocker agent is a benzothiazepine.

19. The medicament of claim 16 wherein said calcium channel blocker agent is a dihydropyridines.

20. The medicament of claim 16 wherein said calcium channel blocker agent is Nifedipine.

21. The medicament of claim 16 wherein said medicament comprises:
verapamil;
a lecithin/isopropyl myristate solution;
butylated hydroxy toluene;
pluronic F127; and
water.

22. The medicament of claim 16 wherein said medicament comprises:
one or more calcium antagonist selected from the calcium channel blocker categories of diphenylalkylamines, benzothiazepines, and dihydropyridines;
a lecithin/isopropyl myristate solution;
butylated hydroxy toluene;
pluronic F127; and
water.

23. The medicament of claim 16 wherein said medicament comprises:
a diphenylalkylamine calcium antagonist agent;
a lecithin/isopropyl myristate solution;
butylated hydroxy toluene;
pluronic F127; and
water.

24. The medicament of claim 21 further comprising:
Edetate disodium.

25. The medicament of claim 22 further comprising:
Edetate disodium.

26. The medicament of claim 23 further comprising:
Edetate disodium.

27. The medicament of claim 24 further comprising:
Propylene glycol.

28. The medicament of claim 25 further comprising:
Propylene glycol.

29. The medicament of claim 26 further comprising:
Propylene glycol.
